(19) United States
(12) Patent Application Publication
(10) Pub. No.: US 2025/0299387 A1
(43) Pub. Date: Sep. 25, 2025

(54) METHODS AND APPARATUS FOR MACHINE LEARNING BASED MEDICAL IMAGING EVENT DETECTION AND IMAGE RECONSTRUCTION

(71) Applicant: Siemens Medical Solutions USA, Inc., Malvern, PA (US)

(72) Inventors: Ziad Burbar, Knoxville, TN (US); Stefan Siegel, Knoxville, TN (US)

(21) Appl. No.: 18/615,078

(22) Filed: Mar. 25, 2024

(51) Int. Cl. G06T 11/00; A61B 6/03; G01T 1/29

(52) U.S. Cl. CPC G06T 11/005; A61B 6/037; G01T 1/2985; G06T 2211/441

(57) ABSTRACT

Systems and methods for detecting multiple events during nuclear imaging scans, and for reconstructing images based on the detected events, are disclosed. In some embodiments, an image scanning system scans a subject, and generates a signal characterizing a detection event. The system generates sampled data based on sampling the at least one signal. Further, the system applies a trained machine learning process to the sampled data. Based on the application of the trained machine learning process, the system generates pulse data characterizing a plurality of decoupled pulses. For example, the pulse data may characterize pulse energy values of each of the decoupled pulses, and corresponding times for each of the pulses. Further, the method includes transmitting the pulse data to generate time-coincident pairs for image reconstruction.

### FIELD

Aspects of the present disclosure relate in general to medical diagnostic systems and, more particularly, to capturing and reconstructing images from nuclear imaging systems for diagnostic and reporting purposes.

### BACKGROUND

Nuclear imaging systems can employ various technologies to capture images. For example, some nuclear imaging systems employ positron emission tomography (PET) to capture images. PET is a nuclear medicine imaging technique that produces tomographic images representing the distribution of positron emitting isotopes within a body. Some nuclear imaging systems combine images from PET and a co-modality, such as computed tomography (CT) or Magnetic Resonance Imaging (MRI). CT is an imaging technique that uses x-rays to produce anatomical images. Magnetic Resonance Imaging (MRI) is an imaging technique that uses magnetic fields and radio waves to generate anatomical and functional images, and may also be used as a co-modality. These nuclear imaging systems can combine images from PET and co-modality scanners during an image fusion process to produce images that show information from both the PET scan and the co-modality scan (e.g., PET/CT systems). Moreover, the nuclear imaging systems may generate an attenuation map that can be used to correct the PET measurement data during image reconstruction.

Typically, PET systems (e.g., time-of-flight (TOF) PET systems) include a scanner with detector elements that include crystals which can detect gamma rays during the scanning process. The detector elements include crystals that detect the gamma rays. The systems can generate measurement data characterizing an image based on these detections. Sometimes, however, these systems ignore or miss detection events when, for instance, multiple detection events occur near each other in time. As a result, the generated measurement data may suffer in accuracy, as well as any medical image reconstructed based on the measurement data. As such, there are opportunities to address these and other deficiencies in nuclear imaging systems.

### SUMMARY

Systems and methods for detecting multiple events during nuclear imaging scans, and for reconstructing medical images based on the detected events, are disclosed.

In some embodiments, a computer-implemented method includes receiving at least one signal characterizing a detection event. The method also includes generating sampled signal data based on sampling the at least one signal. Further, the method includes applying a trained machine learning process to the at least one signal and, based on the application of the trained machine learning process, generating pulse data characterizing a plurality of pulses. The method also includes transmitting the pulse data characterizing the plurality of pulses.

In some embodiments, a non-transitory computer readable medium stores instructions that, when executed by at least one processor, cause the at least one processor to perform operations including receiving at least one signal characterizing a detection event. The operations also include generating sampled signal data based on sampling the at least one signal. Further, the operations include applying a trained machine learning process to the at least one signal and, based on the application of the trained machine learning process, generating pulse data characterizing a plurality of pulses. The operations also include transmitting the pulse data characterizing the plurality of pulses.

In some embodiments, a system includes a memory device storing instructions, a transceiver, and at least one processor communicatively coupled the transceiver and the memory device. The at least one processor is configured to execute the instructions to receive, via the transceiver, at least one signal characterizing a detection event. The at least one processor is also configured to execute the instructions to generate sampled signal data based on sampling the at least one signal. Further, the at least one processor is configured to execute the instructions to apply a trained machine learning process to the at least one signal and, based on the application of the trained machine learning process, generate pulse data characterizing a plurality of pulses. The at least one processor is also configured to execute the instructions to transmit the pulse data characterizing the plurality of pulses.

### BRIEF DESCRIPTION OF THE DRAWINGS

The following will be apparent from elements of the figures, which are provided for illustrative purposes and are not necessarily drawn to scale.

FIGS. 1A, 1B, 1C, and 1D illustrate portions of a nuclear imaging system, in accordance with some embodiments.

FIG. 2 illustrates a block diagram of an example computing device that can perform one or more of the functions described herein, in accordance with some embodiments.

FIG. 3 illustrates a reconstruction of a nuclear image using a neural network, in accordance with some embodiments.

FIG. 4 is a flowchart of an example method to generate data characterizing multiple pulses, in accordance with some embodiments.

FIG. 5 is a flowchart of an example method to train a machine learning process, in accordance with some embodiments.

FIGS. 6A and 6B illustrate exemplary real and analytical pulses, in accordance with some embodiments.

FIG. 7 illustrates two pulses fitted to a pileup pulse, in accordance with some embodiments.

### DETAILED DESCRIPTION

This description of the exemplary embodiments is intended to be read in connection with the accompanying drawings, which are to be considered part of the entire written description. Independent of the grammatical term usage, individuals with male, female, or other gender identities are included within the term.

The exemplary embodiments are described with respect to the claimed systems as well as with respect to the claimed methods. Furthermore, the exemplary embodiments are described with respect to methods and systems for image reconstruction, as well as with respect to methods and systems for training functions used for image reconstruction. Features, advantages, or alternative embodiments herein can be assigned to the other claimed objects and vice versa. For example, claims for the providing systems can be improved with features described or claimed in the context of the methods, and vice versa. In addition, the functional features of described or claimed methods are embodied by objective units of a providing system. Similarly, claims for methods and systems for training image reconstruction functions can be improved with features described or claimed in context of the methods and systems for image reconstruction, and vice versa.

Various embodiments of the present disclosure can employ machine learning methods or processes to provide clinical information from nuclear imaging systems. For example, the embodiments can employ machine learning methods or processes to reconstruct images based on captured measurement data, and provide the reconstructed images for clinical diagnosis. In some embodiments, machine learning methods or processes are trained, to improve the reconstruction of images.

Positron emission tomography (PET) imaging systems can include a scanner with various detection elements. Each detection element may house crystals that detect emitted gamma rays from a scanned subject. When a crystal detects an event, one or more signals may be generated. For example, the system may generate a first signal characterizing an energy associated with the detected event. The system may additionally generate a second signal characterizing a first dimension location of the detection (e.g., X-axis location of the detecting crystal), and a third signal characterizing a second dimension location of the detection (e.g., a Y-axis location of the detecting crystal).

Each of any of these signals (e.g., first signal, second signal, and third signal) may characterize a pulse, where the height of the signal at any given point in time indicates an associated energy value, such as a kilo-electron volts (KeV) value. The energy of a radiation pulse can be measured by the area under the pulse of a particular event. As activity increases in the field-of-view of a systems' scanner, the probability of more than one pulse being generated simultaneously or nearly simultaneously increases. For instance, the scanner may detect a second pulse during a first pulse's duration, or while the scanner is processing the first pulse (e.g., within the pulse processing time of the scanner). Such a situation is referred to as “pileup.” Conventional systems may attempt to detect such circumstances and, if detected, discard the detected event (e.g., do not process the detected event, as if it never happened). The conventional systems may discard such events to prevent introducing errors in energy and detector position (e.g., crystal position) estimates, or because of a lack of timing information associated with such an event. However, discarding such information reduces a system's sensitivity and, as a result, the accuracy of energy and position estimates.

### Using Trained Machine Learning Models to Decouple Pulses

The embodiments described herein employ trained machine learning or artificial intelligence processes that detect pileup events, decouple multiple pulses from the pileup events, and generate energy and position estimates for each of the multiple pulses. For example, a signal received from an imaging scanner may be sampled (e.g., using an analog-to-digital converter (ADC)) to generate a plurality of values. The plurality of values may be inputted to a trained machine learning model (e.g., trained neural network, trained deep learning model, etc.) that is configured to generate output data characterizing whether a pileup event occurred and, if a pileup event occurred, multiple decoupled pulses and a time offset value identifying a time offset between the multiple decoupled pulses. For example, the output data may identify whether the input pulse includes a pileup condition and, if a pileup condition is detected, an energy value for each pulse, and a time between the pulses. Based on the output data, a system may generate energy, position, and time (e.g., timestamp) estimates for each of the multiple pulses.

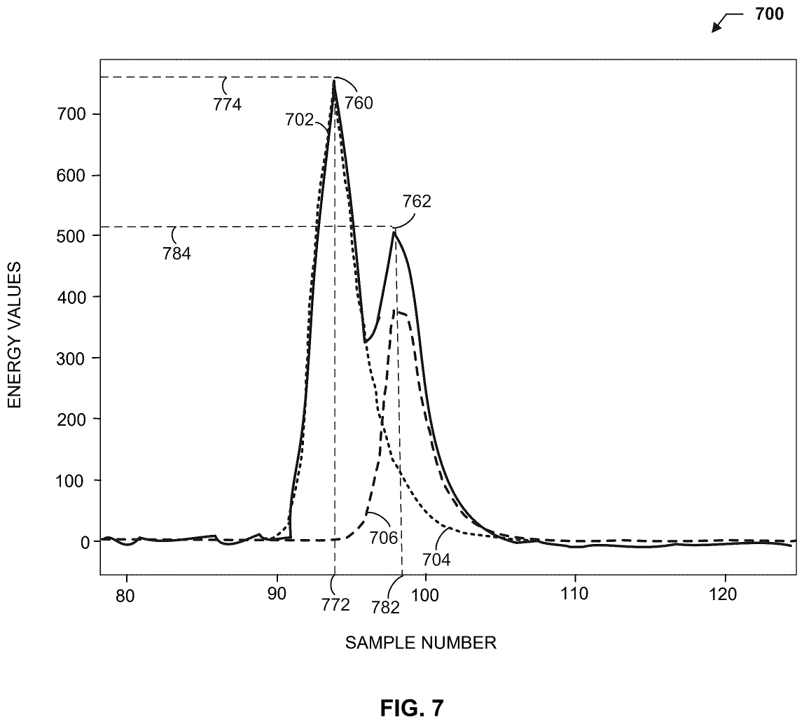

FIG. 7 illustrates a chart 700 that identifies sample numbers (e.g., analog-to-digital converter (ADC) sample numbers) along the X-axis and energy values (e.g., ADC sampled values) along the Y-axis. The chart 700 shows a pileup signal 702 as a solid line, which is a signal that may be received when two events are detected simultaneously or nearly simultaneously, thereby resulting in a pileup event. For instance, the pileup signal 702 may result when one event is detected while the scanner is still processing a signal from a previously detected event (e.g., before the first signal has completely decayed). To decouple two or more pulses from the pileup signal 702, the embodiments described herein may apply the trained machine learning process to the pileup signal 702 to generate output data characterizing two or more individual pulses, such as first decoupled pulse 704 and second decoupled pulse 706.

For instance, the machine learning process may be trained to detect (e.g., classify), based on the pileup signal 702, a first peak 760, which is located at sample number 772 (ADC sample number), and second peak 762, which is located at sample number 782. The trained machine learning process may also detect a first amplitude 774 of the first peak 760 and a second amplitude 784 of the second peak 762. Based on the location and amplitude of each detected peak 760, 762, the trained machine learning process may further generate a corresponding energy value. For example, the energy value may be an area under the curve (AUC) of each respective peak 760, 762. In some examples, the trained machine learning process may determine the energy value for each peak 760, 762 based on detecting a peak value of each peak 760, 762, respectively. As described herein, the output data generated by the trained machine learning process may include the energy values of each detected peak.

Further, the output data of the trained machine learning process may further characterize a time offset value between detected peaks, such as between peaks 760, 762. As an example, assume the sample number 782 of the second peak 762 is 98 and the sample number 772 of the first peak 760 is 93. A time offset value between the first peak 760 and the second peak 762 may be 5 (98−93), where 5 indicates time in terms of the ADC sampling rate. A first time may be assigned to the first decoupled pulse 704 upon pulse detection (e.g., when the signal amplitude reaches a threshold amplitude level), and a second time may be assigned to the second decoupled pulse 706 based on the first time and the time offset value. For instance, the second time may be the offset added to the first time.

The embodiments may perform any of these pileup detection processes for any signals received from a scanner, such as a first signal characterizing energy, and second and third signals characterizing crystal locations. Moreover, and as described herein, the energy values corresponding time values may be used to generate time-coincident pair data for image reconstruction.

### Training the Machine Learning Models

The machine learning processes described herein may be based on, for example, classifier models, decision tree (e.g., Random Forrest) models, neural networks (e.g., convolutional neural network, deep neural network), artificial intelligence models, or any other suitable model, and may be trained with training data characterizing various pulses that simulate pulses out image scanners. For example, pulses with various pulse shapes can be generated based on one or more functions. Function 1, below, illustrates a function Y (t) that characterizes a pulse.

$$Y ( t ) = K s * exp ( ( - 1 * ( t - Ki ) / tf ) ) / ( 1 + exp ( - 1 * ( t - Ki ) / tr ) ) \qquad ( 1 )$$

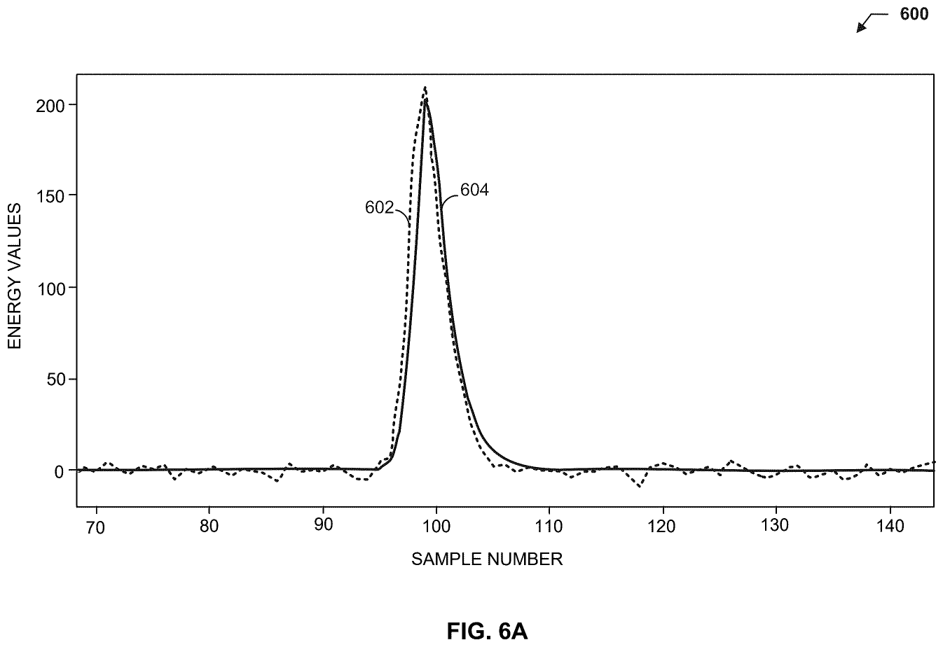

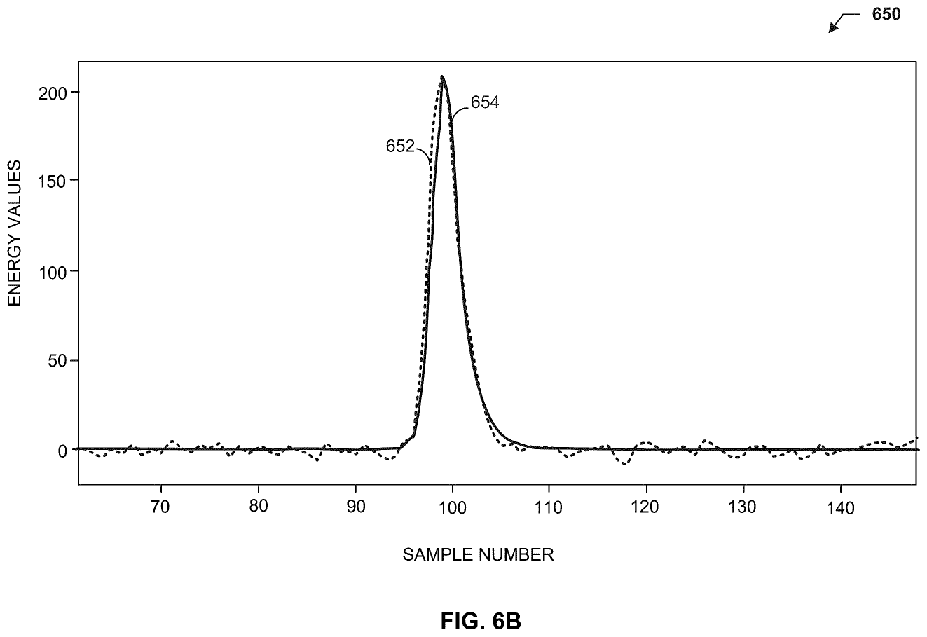

Here, the variable t represents time, the variable Ks is a global scale factor, the variable Ki is a time-offset term, the variable tf is a decay time constant for the pulse, and the variable tr is a rise time constant for the pulse. FIG. 6A, for example, illustrates a chart 600 that identifies sample numbers along the X-axis and energy values (e.g., ADC sampled values) along the Y-axis. The chart 600 includes a captured pulse 602 illustrated in dashed lines overlaid with an analytical pulse 604 illustrated in a solid line. The analytical pulse 604 was computed using function 1 above with corresponding values for Ks, Ki, tf, and tr. Similarly, FIG. 6B illustrates a chart 650 with a captured pulse 652 illustrated in dashed lines overlaid with an analytical pulse 654 illustrated in a solid line. The analytical pulse 654 was also computed using function 1 above with the same values for Ks, Ki, tf, and tr. As indicated by each of these figures, the analytical pulses 604, 654, closely match the captured pulses 602, 652, respectively.

As such, to generate the training data, a synthetic pulse (e.g., data values that characterize a pulse) is generated, for example, according to function 1 above. This synthetic pulse is then phase shifted and sampled to generate additional synthetic pulses. As an example, a synthetic initial pulse may generated according to Function 1 using a predetermined digitizer sample time (e.g., t=1 ns) and predetermined values for the remaining parameters. This synthetic initial pulse is then sampled at a system rate (e.g., 20 nano-seconds) to generate a first synthetic pulse. The system rate may be the rate at which an ADC in the targeted image scanning system samples at. The synthetic initial pulse is then phase shifted by a predetermined amount (e.g., 1 nano-seconds), and is re-sampled at the system rate to generate a second synthetic pulse. This process of phase shifting and sampling is repeated for any number of times, such as until the synthetic initial pulse has been phase shifted up to two times its width.

Further, each of the generated synthetic pulses are added with each other to generate synthetic pileup signals. For example, the first synthetic pulse may be added with the second synthetic pulse to generate a first pileup pulse. Similarly, the first synthetic pulse may be added with a third synthetic pulse to generate a second pileup pulse. In addition, the second synthetic pulse may be added with the third synthetic pulse to generate a third pileup pulse. Additional pileup pulses may be generated by adding pairs of synthetic pulses. In some examples, the amplitude of any of the synthetic pulses may be adjusted (e.g., increased or decreased), before generating corresponding pileup pulses. For instance, the amplitude of the synthetic pulses may be adjusted by 10 KeV to generate additional pileup pulses. These pileup pulses and corresponding synthetic pulses may be used as the training data to train the machine learning processes. For example, each pileup pulse may be labelled as an input, and the corresponding synthetic pulses may be labelled as the expected output. These labelled pileup pulses and corresponding synthetic pulses may then be inputted to the machine learning model being trained. Although described here as adding pairs of pulses, to decouple three or more pulses, the machine learning model may be trained with pileup pulses generated based on a higher number of corresponding synthetic pulses, thereby allowing the trained machine learning process to decouple the higher number of pulses when detecting a pileup event.

### Exemplary Embodiments

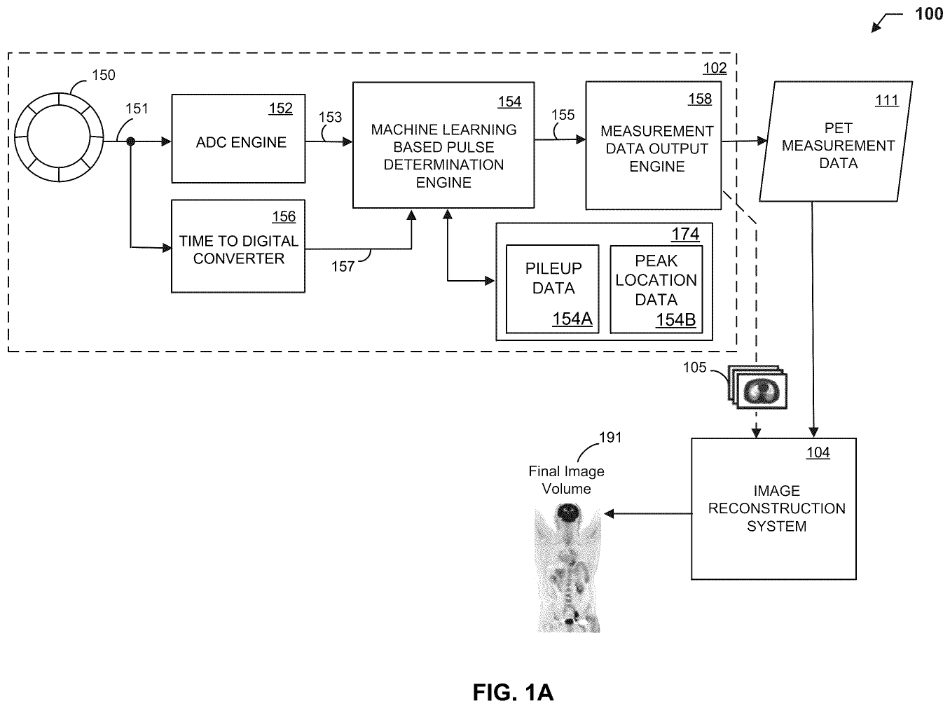

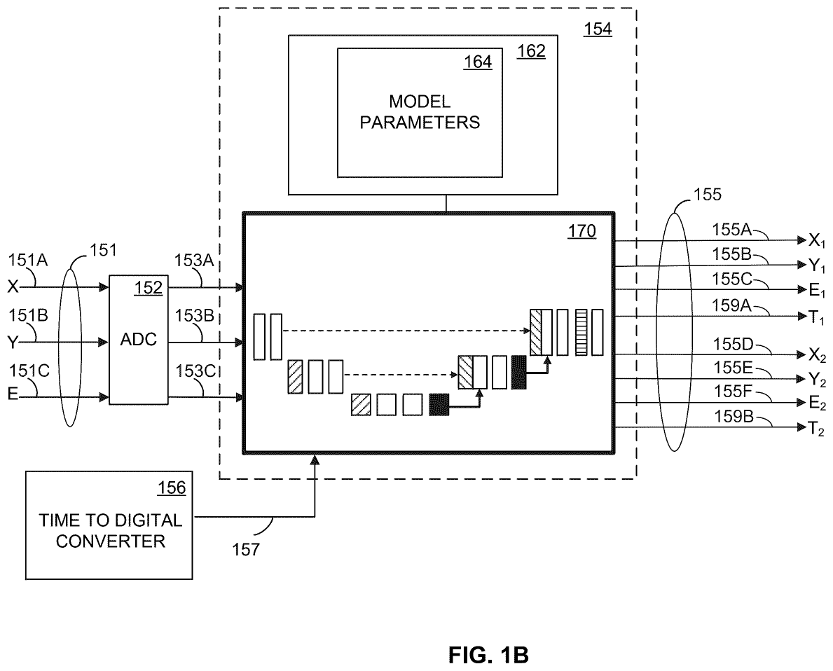

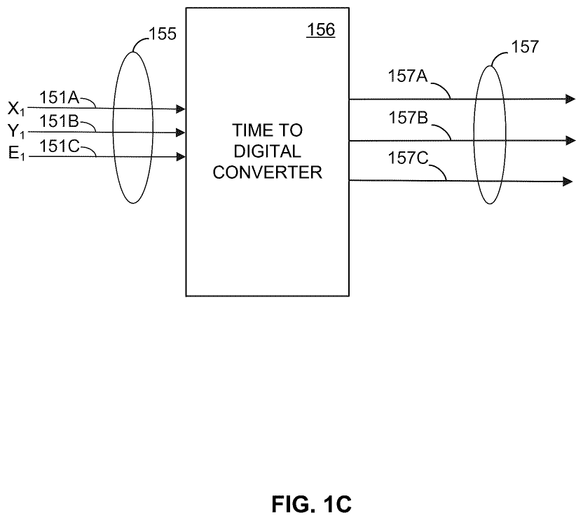

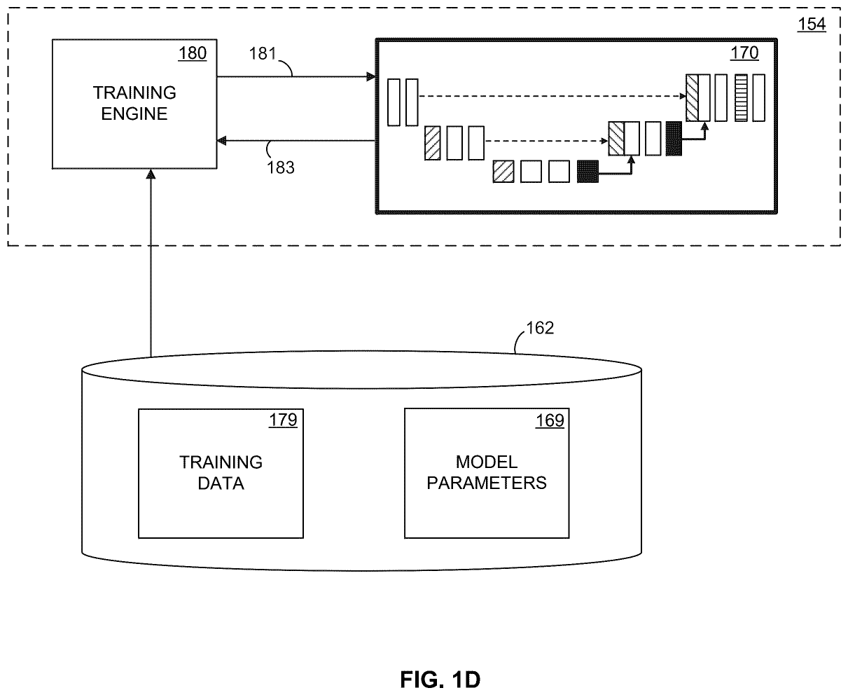

Referring now to FIG. 1A, a nuclear imaging system 100 includes an image scanning system 102 and an image reconstruction system 104. Image scanning system 102 may be a PET scanner that can capture PET images, a PET/MR scanner that can capture PET and MR images, a PET/CT scanner that can capture PET and CT images, or any other suitable image scanner. For example, image scanning system 102 can capture PET images (e.g., of a person), and can generate PET measurement data 111 based on the captured PET images. The PET measurement data 111 (e.g., sinogram data, list-mode data) can represent anything imaged in the scanner's field-of-view (FOV) containing positron emitting isotopes. Moreover, the PET measurement data 111 may identify detection events (e.g., time-coincident pair data) and related timing information. The image scanning system 102 can transmit the PET measurement data 111 to the image reconstruction system 104 for image reconstruction. For example, the image reconstruction system 104 may apply machine learning processes to the PET measurement data to generate a final image volume 191 characterizing a reconstructed image.

In some examples, image scanning system 102 may additionally generate attenuation maps 105 (e.g., u-maps). For instance, the image scanning system 102 may be a PET/CT scanner that, in addition to PET images, can capture CT scans of the patient. The image scanning system 102 may generate the attenuation maps 105 based on the captured CT images, and may transmit the attenuation maps 105 to the image reconstruction system 104. As another example, the image scanning system 102 may be a PET/MR scanner that, in addition to PET images, can capture MR scans of the patient. The image scanning system 102 may generate the attenuation maps 105 based on the captured MR images, and may transmit the attenuation maps 105 to the image reconstruction system 104.

In this example, image scanning system 102 includes a scanner 150, an analog-to-digital-converter (ADC) engine 152, a machine learning based pulse determination engine 154, a time to digital converter (TDC) 156, and a measurement data output engine 158. In some examples, all or parts of image scanning system 102 are implemented in hardware, such as in one or more field-programmable gate arrays (FPGAs), one or more application-specific integrated circuits (ASICs), one or more state machines, one or more computing devices, digital circuitry, or any other suitable circuitry. For example, all or parts of ADC engine 152, machine learning based pulse determination engine 154, TDC 156, and measurement data output engine 158 may be implemented within one or more FPGAs. In some examples, parts or all of image scanning system 102 can be implemented in software as executable instructions such that, when executed by one or more processors, cause the one or more processors to perform respective functions as described herein. The instructions can be stored in a non-transitory, computer-readable storage medium, and can be read and executed by the one or more processors.

The scanner 150 may include detector elements that include crystals which can detect gamma rays during the scanning (e.g., imaging) process. Specifically, for each detection event, scanner 150 may generate detection data 151 that includes one or more signals (e.g., for one or more detection channels). For example, detection data 151 may include a first signal characterizing detected energy levels (e.g., energy depositions), a second signal characterizing a first dimension position (e.g., X-axis position) of the detecting crystal, and a third signal characterizing a second dimension position (e.g., Y-axis position) of the detecting crystal. The scanner 150 may provide the detection data 151 characterizing the detected events to ADC engine 152.

ADC engine 152 may include an analog-to-digital converter (ADC) that samples each signal characterized by the detection data 151 at a sample rate to generate ADC data 153 characterizing the sampled signal. For example, ADC engine 152 may sample each signal at a Nyquist rate, such as every 20 nano-seconds, and, based on the sampling, may generate ADC data 153 characterizing corresponding voltage levels for the signal. ADC engine 152 may transmit the ADC data 153 for each signal characterized by the detection data 151 to machine learning based pulse determination engine 154.

The machine learning based pulse determination engine 154 may receive the ADC data 153, and input the ADC data 153 to a trained machine learning process that, in response to the inputted ADC data 153, generates and outputs pulse data 155 characterizing each detected event. For example, trained machine learning process may determine that the sampled signal data includes a pileup event, and generates and outputs pulse data 155 characterizing each detected event. In other words, the machine learning process may be trained to detect, based on ADC data 153, two or more pulses that, when added to each other, form the sampled signal characterized by ADC data 153, and may generate pulse data 155 characterizing the two or more detected pulses. For instance, if no pileup event is detected, the pulse data 155 may characterize one pulse corresponding to one detected event. If, however, a pileup event was detected, the pulse data 155 may characterize two or more pulses, each pulse corresponding to a detected event.

In some examples, machine learning based pulse determination engine 154 may generate pileup data 154A indicating whether or not the ADC data 153 includes a pileup event (e.g., two or more pulses) for each signal characterized by the detection data 151. Additionally or alternatively, machine learning based pulse determination engine 154 may generate peak location data 154B identifying a location of each detected peak for each of the signals. For example, the peak location data 154B may identify an ADC sample number, or a range of ADC sample numbers, of the ADC data 153 corresponding to each detected peak. Machine learning based pulse determination engine 154 may store the pileup data 154A and peak location data 154B within a memory device 174, for example.

In some examples, the pulse data 155 may characterize energy values. As described herein, the energy value may be an area-under-the curve (AUC) value or peak value for each detected pulse. For example, if a pileup event is detected, the pulse data 155 generated by the trained machine learning process may include energy values characterizing each of the decoupled pulses (e.g., an energy value for the first decoupled pulse 704, and an energy value for the second decoupled pulse 706). For instance, the trained machine learning process may be trained to output the energy values included in the pulse data 155 for each decoupled pulse. In some examples, the machine learning based pulse determination engine 154 may execute function 1 based on corresponding pulse parameters for each pulse decoupled by the trained machine learning process to determine an energy value Y (t) at each peak location. In some examples, the machine learning based pulse determination engine 154 determines an event occurred when the energy value is within a range, such as above a lower discriminator and below an upper discriminator. If the energy value is not within the range, the machine learning based pulse determination engine 154 may discard (e.g., ignore) the event.

As an example, the machine learning based pulse determination engine 154 may receive detection data 151 from ADC engine 152. The detection data 151 may characterize three signals, such as an energy signal, a first dimension location signal, and a second dimension location signal. Machine learning based pulse determination engine 154 may apply the trained machine learning process to each of the three signals to detect two pulses on each of the three signals, and may further determine energy values at the locations of the peaks of each of the two pulses on each of the three signals. Machine learning based pulse determination engine 154 may generate pulse data 155 characterizing the energy values for each of the two pulses on each of the three signals.

As illustrated, machine learning based pulse determination engine 154 may also receive time data 157 from TDC 156. The time data 157 may characterize a time associated with each detection event received for the one or more signals of the detection data 151. For instance, TDC 156 may receive the detection data 151, and generate, for each detection event of each signal of the detection data 151, time data 157 characterizing a digital time. For example, in response to receiving a detection event on a signal of detection data 151, TDC 156 may sample a system time and, based on the sampled system time, generate time data 157 for the signal. As described herein, for instance, detection data 151 may characterize events detected for one or more signals (e.g., one or more detection channels). In response to each received event, TDC 156 may generate time data 157 for that event. TDC 156 may transmit the time data 157 to machine learning based pulse determination engine 154.

Machine learning based pulse determination engine 154 may receive the time data 157 and, based on the time data 157, determine a time for each pulse detected on the corresponding signal of the detection data 151. For instance, machine learning based pulse determination engine 154 may generate a first time for a first pulse (earlier pulse) based on the time data 157, and may generate the second time for a second pulse (later pulse) based on the first time and a corresponding time offset value (e.g., peak location data 154B). In an example where the time offset value represents a difference in sample numbers, machine learning based pulse determination engine 154 may scale the time offset value to the system time (e.g., to same units as the system time), and add the scaled offset value to the first time to generate the second time. In addition to characterizing energy values for one or more detected pulses, machine learning based pulse determination engine 154 may generate the pulse data 155 to characterize corresponding times (e.g., timestamps) for each pulse on each signal. For instance, pulse data 155 may include pulse-time pairs, where each pulse-time pair characterizes a detected pulse and a corresponding time associated with the detected pulse. Machine learning based pulse determination engine 154 may transmit the pulse data 155 to measurement data output engine 158.

Measurement data output engine 158 may receive the pulse data 155 from the machine learning based pulse determination engine 154. Based on the pulse data 155, measurement data output engine 158 may generate PET measurement data 111. The PET measurement data 111 identifies detected crystal pulses (e.g., time-coincident data pairs) as well as corresponding times (e.g., time offsets between detection events). For example, the measurement data output engine 158 may perform processes to generate time-coincident pairs based on the times and pulses identified by the pulse data 155, and may generate the PET measurement data 111 to include the determined time-coincident pairs.

Further, and as illustrated, image reconstruction system 104 may receive the PET measurement data 111 from the image scanning system 102. Image reconstruction system 104 may perform operations to reconstruct an image based on the PET measurement data 111, and may generate final image volume 191 characterizing the reconstructed image. For example, image reconstruction system 104 may apply one or more trained machine learning processes to the PET measurement data 111 and, based on applying the one or more trained machine learning processes, may generate the final image volume 191. In some instances, image reconstruction system 104 receives an attenuation map 105 from the image scanning system 102. The image reconstruction system 104 may perform operations to correct the PET measurement data 111, for example for attenuation, based on the attenuation map 105. For instance, the image reconstruction system 104 may apply one or more attenuation correction processes to the output of the trained machine learning process and the attenuation map 105 and, based on applying the one or more attenuation correction processes, may generate the final image volume 191.

Image reconstruction system 104 may transmit the final image volume 191 characterizing the reconstructed image. For instance, image reconstruction system 104 and may transmit the final image volume 191 for display, and/or may store the final image volume within a data repository.

FIG. 1B illustrates exemplary portions of image scanning system 102 including an example of ADC engine 152 and machine learning based pulse determination engine 154, an executed and trained machine learning model 170, and a memory 162. Memory 162 may store model parameters 164 (e.g., hyperparameters, weights, etc.) characterizing the trained machine learning model 170. Machine learning based pulse determination engine 154 may establish the trained machine learning model 170 based on the model parameters 164. For example, machine learning based pulse determination engine 154 may obtain the model parameters 164 from the memory 162, and may execute the trained machine learning model 170 based on the model parameters 164.

As illustrated, detection data 151 may include three signals, including a first dimension location signal 151A, a second dimension location signal 151B, and an energy signal 151C. The first dimension location signal 151A may characterize a first dimension of a location of a crystal detecting an event (e.g., an X axis location), while the second dimension location signal 151B may characterize a second dimension of the location of the crystal detecting the event (e.g., a Y axis location). Further, the energy signal 151C may characterize energy (e.g., energy levels) of the detected event. ADC engine 152 may sample each of the first dimension location signal 151A, second dimension location signal 151B, and energy signal 151C at a particular rate (e.g., every 20 nano-seconds), and may generate first dimension ADC data 153A, second dimension ADC data 153B, and energy ADC data 153C, respectively.

Machine learning based pulse determination engine 154 may input the first dimension ADC data 153A, second dimension ADC data 153B, and energy ADC data 153C to the executed and trained machine learning model 170 and, in response, the executed and trained machine learning model 170 may generate pulse data 155 characterizing detected pulses.

For instance, based on applying the executed and trained machine learning model 170 to the first dimension location signal 151A, the executed and trained machine learning model 170 may detect whether the first dimension location signal 151A includes a pileup event. For instance, if no pileup event is detected, the executed and trained machine learning model 170 may output first pulse first dimension location 155A characterizing an energy value for one detected pulse (e.g., one event). In addition, based on corresponding time data 157 received from TDC 156, the executed and trained machine learning model 170 may determine an associated time of the event, and may generate first time data 159A characterizing the determined time. As such, the first time data 159A generated by the executed and trained machine learning model 170 characterizes a corresponding time (e.g., timestamp) associated with the first pulse first dimension location 155A.

If, however, a pileup event is detected, the executed and trained machine learning model 170 may output pulse data 155 charactering energy values for multiple detected events. For instance, and assuming the executed and trained machine learning model 170 detects two pulses, the executed and trained machine learning model 170 may generate and output first pulse first dimension location 155A characterizing a first energy value for a first detected pulse, and may generate and output second pulse first dimension location 155D characterizing a second energy value for a second detected pulse. Further, and as described herein, based on corresponding time data 157 received from TDC 156, the executed and trained machine learning model 170 determine a first time for the first detected pulse, and may generate first time data 159A characterizing the first time. The executed and trained machine learning model 170 may also determine a time offset between the first detected pulse and the second detected pulse, and determine a second time for the second detected pulse based on the first time and the time offset. The executed and trained machine learning model 170 may generate second time data 159B characterizing the second time associated with the second pulse first dimension location 155D.

Similarly, based on applying the executed and trained machine learning model 170 to the second dimension location signal 151B, the executed and trained machine learning model 170 may detect whether the second dimension location signal 151B includes a pileup event. For instance, if no pileup event is detected, the executed and trained machine learning model 170 may output first pulse second dimension location 155B characterizing an energy value for one detected pulse (e.g., one event). In addition, as described herein, the executed and trained machine learning model 170 may determine an associated time of the event based on the corresponding time data 157 received from TDC 156, and may generate the first time data 159A characterizing the determined time. As such, the first time data 159A generated by the executed and trained machine learning model 170 characterizes a corresponding time (e.g., timestamp) associated with the first pulse second dimension location 155B.

If, however, a pileup event is detected, the executed and trained machine learning model 170 may output pulse data 155 charactering energy values for multiple detected events. For instance, and assuming the executed and trained machine learning model 170 detects two pulses, the executed and trained machine learning model 170 may generate and output first pulse second dimension location 155B characterizing a first energy value for a first detected pulse, and may generate and output second pulse second dimension location 155E characterizing a second energy value for a second detected pulse. Further, and as described herein, based on corresponding time data 157 received from TDC 156, the executed and trained machine learning model 170 may generate first time data 159A characterizing a first time for the second dimension location signal 151B, and second time data 159B characterizing a second time for the second pulse second dimension location 155E.

Further, and based on applying the executed and trained machine learning model 170 to the energy signal 151C, the executed and trained machine learning model 170 may detect whether the energy signal 151C includes a pileup event. For instance, if no pileup event is detected, the executed and trained machine learning model 170 may output first pulse energy value 155C characterizing an energy value for one detected pulse (e.g., one event). In addition, as described herein, the executed and trained machine learning model 170 may determine an associated time of the event based on corresponding time data 157 received from TDC 156, and may generate the first time data 159A characterizing the determined time. As such, in this example, first time data 159A characterizes a time (e.g., timestamp) associated with an event causing the first dimension location signal 151A, the second dimension location signal 151B, and the energy signal 151C, and the second time data 159B.

If, however, a pileup event is detected, the executed and trained machine learning model 170 may output pulse data 155 charactering energy values for multiple detected events. For instance, and assuming the executed and trained machine learning model 170 detects two pulses, the executed and trained machine learning model 170 may generate and output first pulse energy value 155C characterizing a first energy value for a first detected pulse, and may generate and output second pulse energy value 155F characterizing a second energy value for a second detected pulse. Further, and as described herein, based on corresponding time data 157 received from TDC 156, the executed and trained machine learning model 170 may generate first time data 159A characterizing a first time for the first pulse energy value 155C, and second time data 159B characterizing a second time for the second pulse energy value 155F. As such, in this example, second time data 159B characterizes a time (e.g., timestamp) associated with the generated second pulses, namely, second pulse first dimension location 155D, second pulse second dimension location 155E, and second pulse energy value 155F.

FIG. 1B is illustrated and described above with respect to decoupling two pulses on each of three signals merely for ease of readability reasons. In other embodiments as contemplated herein, three, four, or more pulses may be detected and decoupled from any number of received signals, such as from any of first dimension location signal 151A, second dimension location signal 151B, and the energy signal 151C.

FIG. 1C illustrates an example of the TDC 156. In this example, TDC 156 receives first dimension location signal 151A, second dimension location signal 151B, and energy signal 151C from, for example, the scanner 150. In response to receiving each of the first dimension location signal 151A, second dimension location signal 151B, and energy signal 151C, the TDC 156 generates corresponding time data 157, namely, first dimension time data 157A, second dimension time data 157B, and energy time data 157C. In some instances, to generate the first dimension time data 157A, second dimension time data 157B, and energy time data 157C, TDC 156 may sample a system time (e.g., system time of the image scanning system 102). TDC 156 receives the pulse data 155 (e.g., generated by machine learning based pulse determination engine 154.

FIG. 1D illustrates the training of the executed machine learning model 170 that, for example, can be implemented by the machine learning based pulse determination engine 154. As illustrated, a training engine 180 receives training data 179 from memory 162. The training data can include synthetic signals (e.g., data values that characterize a generated signal).

For example, a synthetic signal may be generated based on sampling a signal from an image scanner, such as a signal of detection data 151 received from scanner 150. The signal may be sampled at a rate corresponding to the image scanning system's 102 sampling rate (e.g., 20 nano-seconds). As an alternative, a synthetic initial pulse may be generated based on Function 1 using a predetermined digitizer sample time (e.g., t=1 ns) and predetermined values for the remaining parameters. This synthetic initial pulse is then sampled at a system rate (e.g., 20 nano-seconds) to generate the synthetic signal. The synthetic signal may then phase shifted and then sampled to generate additional synthetic signals. For instance, the synthetic signal may be phase shifted in increments of a predetermined amount (e.g., 1 nano-seconds), and sampled at each increment, to generate the additional synthetic signals. In addition, pileup synthetic signals may be generated based on combining the generated synthetic signals. For instance, the synthetic signals may be added with one another (e.g., in pairs) to generate corresponding pileup synthetic signals. The training data 179 may include sets of pileup synthetic signals and corresponding pileup synthetic signals (e.g., the pileup synthetic signals used to generate each pileup synthetic signal).

Referring back to FIG. 1D, the training engine 180 obtains a portion of the training data 179 (e.g., a first set of the training data 179), generates features (e.g., feature vectors) based on the obtained portion of the training data 179, and inputs the generated features 181 to the executed machine learning model 170. In some examples, training engine 180 labels the pileup synthetic signals as inputs, and the corresponding synthetic signals as the expected output, e.g., during supervised learning.

After initially training the executed machine learning model 170 with the portion of the training data 179, the training engine 180 may generate additional features based on another portion of the training data 179 (e.g., a second set of the training data 179, the second set being distinct, in some examples, from the first set), and may input the additional features 181 (e.g., without labelling) to the executed machine learning model 170. In response, the training engine 180 may receive output data 183 from the executed machine learning model 170. The output data may characterize decoupled signals (e.g., decoupled pulses).

Further, the training engine 180 may determine whether training is complete based on the output data 183. For example, the training engine 180 may determine one or more metric values based on the output data 183 and expected output data. The metric may be, for instance, a loss function, such as computed precision values, computed recall values, a computed AUC value, any receiver operating characteristic (ROC) curve or precision-recall (PR) curve value, or any other suitable metric. The training engine 180 may determine that training is complete when the one or more metrics satisfy one or more corresponding thresholds. If training is not complete (e.g., one or more metrics did not satisfy their one or more corresponding thresholds), the training engine 180 continues training the executed machine learning model 170 with labelled synthetic signals until computed metrics satisfy their corresponding thresholds.

Once training is complete, the training engine 180 reads model parameters 169 from the executed machine learning model 170, and stores the model parameters 169 within any suitable data repository, such as memory 162. The model parameters 169 may be used to configure the trained machine learning model and establish a trained machine learning process, as described herein.

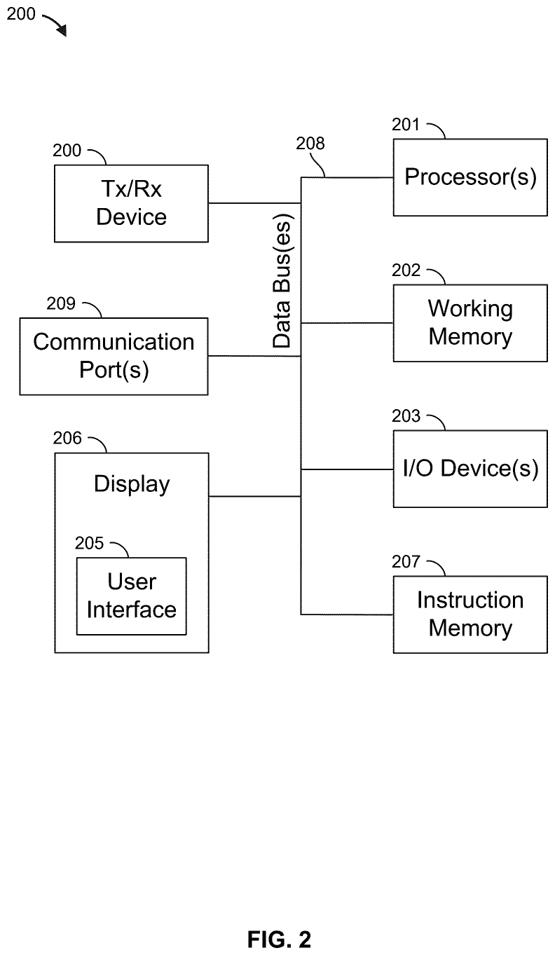

FIG. 2 illustrates a computing device 200 that can be employed by any of the image scanning system 102 and the image reconstruction system 104. For instance, computing device 200 can implement one or more of the functions of the image scanning system 102 and/or image reconstruction system 104 described herein.

Computing device 200 can include one or more processors 201, working memory 202, one or more input-output devices 203, instruction memory 207, a transceiver 204, one or more communication ports 209, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Processors 201 can be configured to perform a certain function or operation by executing code, stored on instruction memory 207, embodying the function or operation. For example, processors 201 can be configured to perform one or more of the functions, methods, or operations disclosed herein.

Instruction memory 207 can be any memory device that can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. For example, instruction memory 207 can store instructions that, when executed by one or more processors 201, cause one or more processors 201 to perform one or more of the functions of image reconstruction system 104, such as one or more of the histo-image generation processes, the multi-view attenuation histo-image generation processes, and/or the machine learning processes described herein.

Processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of computing device 200. Working memory 202 can be a random access memory (RAM) such as a static random access memory (SRAM) or dynamic random access memory (DRAM), or any other suitable memory.

Input-output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 209 allows for the programming of executable instructions in instruction memory 207. In some examples, communication port(s) 209 allow for the transfer (e.g., uploading or downloading) of data, such as PET measurement data 111 and/or attenuation maps 105.

Display 206 can display user interface 205. User interface 205 can enable user interaction with computing device 200. For example, user interface 205 can be a user interface for an application that allows for the viewing of final image volumes 191. In some examples, a user can interact with user interface 205 by engaging input-output devices 203. In some examples, display 206 can be a touchscreen, where user interface 205 is displayed on the touchscreen.

Transceiver 204 allows for communication with a network, such as a Wi-Fi network, an Ethernet network, a cellular network, or any other suitable communication network. For example, if operating in a cellular network, transceiver 204 is configured to allow communications with the cellular network. Processor(s) 201 is operable to receive data from, or send data to, a network via transceiver 204.

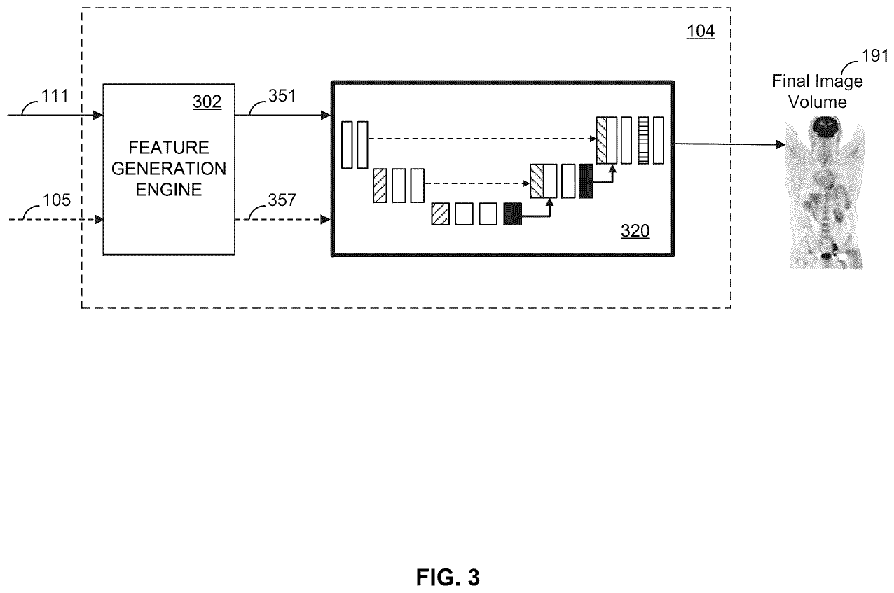

FIG. 3 illustrates exemplary portions of an image reconstruction system 104 that includes a feature generation engine 302 and a trained neural network 320. Image reconstruction system 104 may input the PET measurement data 111 to the feature generation engine 302. In response, feature generation engine 302 may generate detection features 351 based on the PET measurement data 111. Image reconstruction system 104 inputs the detection features 351 to the trained neural network 320. Based on the inputted detection features 351, the trained neural network 320 generates the final image volume 191.

In some examples, the feature generation engine 302 receives an attenuation map 105, and generates map features 357 based on the attenuation map 105. Image reconstruction system 104 may input the map features 357 to the trained neural network 320, and the trained neural network 320 may generate the final image volume 191 based on the inputted detection features 351 and map features 357.

Although the image reconstruction system 104 of FIG. 3 illustrates the generation of the final image volume 191 based on trained machine learning based processes, in other examples, image reconstruction system 104 may generate final image volumes 191 based on other processes, such as by applying analytical or iterative image reconstruction techniques to the PET measurement data 111 to generate the final image volume 191.

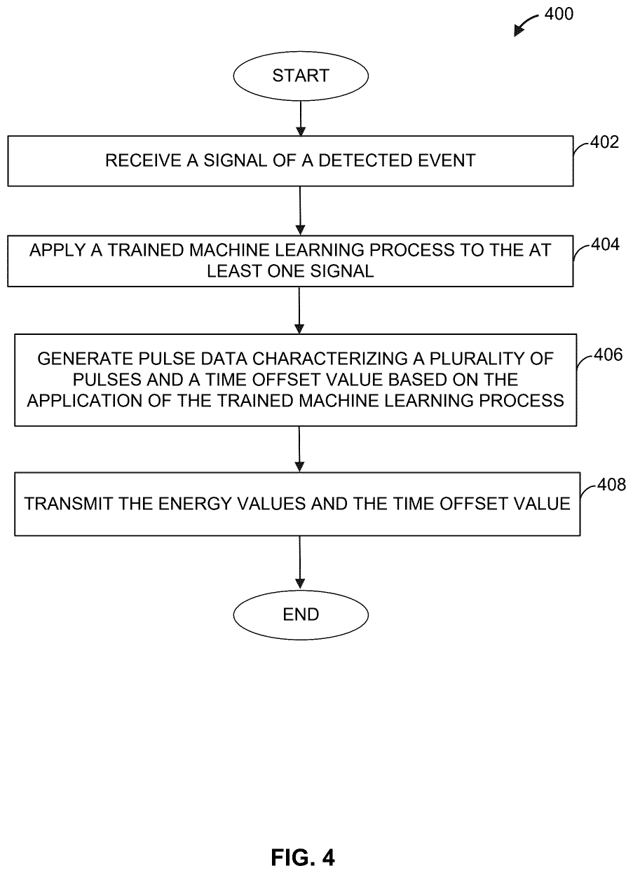

FIG. 4 is a flowchart of an example method 400 to generate data characterizing multiple pulses. The method can be performed by one or more computing devices, such as computing device 200, executing corresponding instructions.

Beginning at block 402, a signal of a detected event is received. For instance, as described herein, scanner 150 of the image scanning system 102 may scan a subject in its field-of-view, and may generate detection data 151 characterizing detection events. The detection data 151 may include, for a given crystal detection, an energy signal, a first location signal characterizing a first dimension location of the detection (e.g., X-axis location of the detecting crystal), and a second location signal characterizing a second dimension location of the detection (e.g., Y-axis location of the detecting crystal).

At block 404, a trained machine learning process is applied to the at least one signal. Further, at block 406, pulse data is generated based on the application of the trained machine learning process to the at least one signal. The pulse data characterizes a plurality of pulses.

For example, as described herein, image scanning system 102 may sample the detection data 151 to generate sampled signal data, and may apply the executed and trained machine learning model 170 to the sampled signal data. In response, the executed and trained machine learning model 170 may generate pulse data 155 that includes, for example, energy values for multiple pulses (e.g., first pulse first dimension location 155A, second pulse first dimension location 155D). The executed and trained machine learning model 170 may also generate a time offset value between the pulses (e.g., first dimension time offset value 155J).

Further, and at block 408, the energy values and time offset value are transmitted. For example, and as described herein, the energy values and time offset value may be transmitted to TDC 156 to establish times for each detected event.

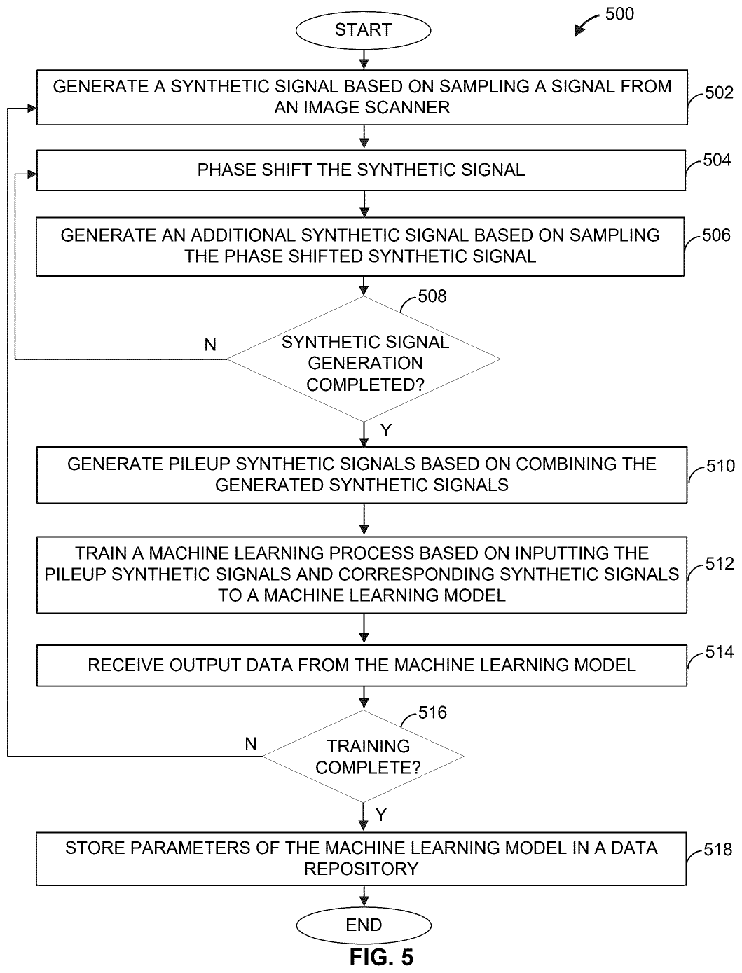

FIG. 5 is a flowchart of an example method 500 to train a machine learning process. The method can be performed by one or more computing devices, such as computing device 200, executing corresponding instructions.

Beginning at block 502, a synthetic signal is generated based on sampling a signal from an image scanner. For example, computing device 200 may receive detection data 151 from scanner 150, and may sample the detection data 151 at, for example, a rate corresponding to the image scanning system's 102 sampling rate (e.g., 20 nano-seconds). As an alternative, a synthetic initial pulse may be generated based on Function 1 using a predetermined digitizer sample time (e.g., t=1 ns) and predetermined values for the remaining parameters. This synthetic initial pulse is then sampled at a system rate (e.g., 20 nano-seconds) to generate the synthetic signal.

At block 504, the synthetic signal is phase shifted. For instance, the synthetic signal may be phase shifted by a predetermined amount (e.g., 1 nano-seconds). Once phase shifted, at block 506, an additional synthetic signal is generated based on sampling the phase shifted synthetic signal. At block 508, a determination is made as to whether synthetic signal generation is complete. For instance, synthetic signal generation may be complete when the synthetic signal has been sampled and phase shifted up to two times its width. If synthetic signal generation is not complete, the method proceeds back to block 504 to continue synthetic signal generation. Otherwise, if synthetic signal generation is complete, the method proceeds to block 510.

At block 510, pileup synthetic signals are generated based on combining the generated synthetic signals. For instance, the synthetic signals generated at blocks 502 and 506 may be added with one another (e.g., in pairs) to generate corresponding pileup synthetic signals. At block 512, a machine learning process is trained based on inputting the pileup synthetic signals and corresponding synthetic signals to a machine learning model. For example, the computing device 200 may label the pileup synthetic signals as inputs, and the corresponding synthetic signals as the expected output, e.g., during supervised learning.

Further, at block 514, additional pileup synthetic signals are inputted (e.g., during validation) to the machine learning model and, in response, output data is received from the machine learning model. The output data may characterize decoupled signals (e.g., decoupled pulses). The additional pileup synthetic signals may be generated in accordance with blocks 502 through 512.

At block 516, a determination is made as to whether training is complete. For example, computing device 200 may determine one or more metric values based on the output data received from the machine learning model and expected output data. The metric may be, for instance, a loss function, such as computed precision values, computed recall values, a computed AUC value, any ROC curve or PR curve value, or any other suitable metric. Computing device 200 may determine that training is complete when the one or more metrics satisfy one or more corresponding thresholds. If training is not complete (e.g., one or more metrics did not satisfy their one or more corresponding thresholds), the method proceeds back to block 502 to continue training the machine learning model. otherwise, if training is complete, the method proceeds to block 518.

At block 518, parameters of the machine learning model are stored in a data repository. For example, the parameters may be stored as model parameters 169 within memory 162. The parameters of the machine learning model may be used to establish a trained machine learning process, as described herein.

The following is a list of non-limiting illustrative embodiments disclosed herein:

Illustrative Embodiment 1: A computer-implemented method comprising:
receiving at least one signal characterizing a detection event;
generating sampled signal data based on sampling the at least one signal;
applying a trained machine learning process to the sampled signal data and, based on the application of the trained machine learning process, generating pulse data characterizing a plurality of pulses; and
transmitting the pulse data characterizing the plurality of pulses.

Illustrative Embodiment 2: The computer-implemented method of illustrative embodiment 1, wherein the pulse data comprises an energy value for each of the plurality of pulses.

Illustrative Embodiment 3: The computer-implemented method of illustrative embodiment 2, wherein the pulse data comprises a time offset value characterizing a time offset between the plurality of pulses.

Illustrative Embodiment 4: The computer-implemented method of illustrative embodiment 3, further comprising:
generating a first time for a first pulse of the plurality of pulses based on sampling a system time; and
generating a second time for a second pulse of the plurality of pulses based on the first time and the time offset.

Illustrative Embodiment 5: The computer-implemented method of any of illustrative embodiments 3-4, further comprising generating image measurement data based on the energy value for each of the plurality of pulses and the time offset value characterizing the time offset between the plurality of pulses.

Illustrative Embodiment 6: The computer-implemented method of illustrative embodiment 5, wherein the image measurement data is positron emission tomography (PET) measurement data.

Illustrative Embodiment 7: The computer-implemented method of any of illustrative embodiments 1-6, wherein applying the trained machine learning process to the sampled signal data comprises:
reading model parameters from a memory device;
executing a machine learning model based on the model parameters; and
inputting the sampled signal data to the executed machine learning model.

Illustrative Embodiment 8: The computer-implemented method of illustrative embodiment 7, further comprising:
generating synthetic signals;
training the machine learning model based on the synthetic signals;
reading the model parameters from the trained machine learning model; and
storing the model parameters in the memory device.

Illustrative Embodiment 9: The computer-implemented method of illustrative embodiment 8, wherein the machine learning model is a Random Forrest model.

Illustrative Embodiment 10: The computer-implemented method of any of illustrative embodiments 1-9, wherein the plurality of pulses consists of two pulses.

Illustrative Embodiment 11: The computer-implemented method of any of illustrative embodiments 10, further comprising receiving the at least one signal from a scanner of an image scanning system.

Illustrative Embodiment 12: The computer-implemented method of any of illustrative embodiments 1-11, wherein the at least one signal comprises a first signal that characterizes energy levels of the detection event.

Illustrative Embodiment 13: The computer-implemented method of illustrative embodiment 12, wherein the at least one signal comprises a second signal that characterizes a first dimension location of a crystal that detected the detection event.

Illustrative Embodiment 14: The computer-implemented method of illustrative embodiment 13, wherein the at least one signal comprises a third signal that characterizes a second dimension location of the crystal that detected the detection event.

Illustrative Embodiment 15: A non-transitory computer readable medium storing instructions that, when executed by at least one processor, cause the at least one processor to perform operations comprising:
receiving at least one signal characterizing a detection event;
generating sampled signal data based on sampling the at least one signal;
applying a trained machine learning process to the sampled signal data and, based on the application of the trained machine learning process, generating pulse data characterizing a plurality of pulses; and
transmitting the pulse data characterizing the plurality of pulses.

Illustrative Embodiment 16: The non-transitory computer readable medium of illustrative embodiment 15, wherein the pulse data comprises an energy value for each of the plurality of pulses.

Illustrative Embodiment 17: The non-transitory computer readable medium of illustrative embodiment 16, wherein the pulse data comprises a time offset value characterizing a time offset between the plurality of pulses.

Illustrative Embodiment 18: The non-transitory computer readable medium of illustrative embodiment 17, wherein the pulse data comprises an energy value for each of the plurality of pulses and a time offset value characterizing a time offset between the plurality of pulses, and wherein the instructions, when executed by the at least one processor, further cause the at least one processor to perform operations comprising:
generating a first time for a first pulse of the plurality of pulses based on sampling a system time; and
generating a second time for a second pulse of the plurality of pulses based on the first time and the time offset.

Illustrative Embodiment 19: The non-transitory computer readable medium of any of illustrative embodiments 17-18, wherein the instructions, when executed by the at least one processor, further cause the at least one processor to perform operations comprising generating image measurement data based on the energy value for each of the plurality of pulses and the time offset value characterizing the time offset between the plurality of pulses.

Illustrative Embodiment 20: The non-transitory computer readable medium of illustrative embodiment 19, wherein the image measurement data is positron emission tomography (PET) measurement data.

Illustrative Embodiment 21: The non-transitory computer readable medium of any of illustrative embodiments 15-20, wherein the instructions, when executed by the at least one processor, further cause the at least one processor to perform operations comprising:
reading model parameters from a memory device;
executing a machine learning model based on the model parameters; and
inputting the sampled signal data to the executed machine learning model.

Illustrative Embodiment 22: The non-transitory computer readable medium of illustrative embodiment 21, wherein the instructions, when executed by the at least one processor, further cause the at least one processor to perform operations comprising:
generating synthetic signals;
training the machine learning model based on the synthetic signals;
reading the model parameters from the trained machine learning model; and
storing the model parameters in the memory device.

Illustrative Embodiment 23: The non-transitory computer readable medium of illustrative embodiment 22, wherein the machine learning model is a Random Forrest model.

Illustrative Embodiment 24: The non-transitory computer readable medium of any of illustrative embodiments 15-23, wherein the plurality of pulses consists of two pulses.

Illustrative Embodiment 25: The non-transitory computer readable medium of any of illustrative embodiments 24, wherein the instructions, when executed by the at least one processor, further cause the at least one processor to perform operations comprising receiving the at least one signal from a scanner of an image scanning system.

Illustrative Embodiment 26: The non-transitory computer readable medium of any of illustrative embodiments 15-25, wherein the at least one signal comprises a first signal that characterizes energy levels of the detection event.

Illustrative Embodiment 27: The non-transitory computer readable medium of illustrative embodiment 26, wherein the at least one signal comprises a second signal that characterizes a first dimension location of a crystal that detected the detection event.

Illustrative Embodiment 28: The non-transitory computer readable medium of illustrative embodiment 27, wherein the at least one signal comprises a third signal that characterizes a second dimension location of the crystal that detected the detection event.

Illustrative Embodiment 29: A system comprising:
a memory device storing instructions;
a transceiver; and
at least one processor communicatively coupled to the transceiver and to the memory device, the at least one processor configured to execute the instructions to:
   receive, via the transceiver, at least one signal characterizing a detection event;
   apply a peak detection process to the at least one signal and, based on the application of the peak detection process, detect a position of each of at least two peaks of the at least one signal;
   determine an amplitude of each of the at least two peaks of the at least one signal;
   apply a curve fitting process to the position and the amplitude of each of the at least two peaks and, based on the application of the curve fitting process, determine an energy value for each of the at least two peaks; and
   transmit, via the transceiver, the energy value for each of the at least two peaks.

Illustrative Embodiment 30: The system of illustrative embodiment 29, wherein the pulse data comprises an energy value for each of the plurality of pulses.

Illustrative Embodiment 31: The system of illustrative embodiment 30, wherein the pulse data comprises a time offset value characterizing a time offset between the plurality of pulses.

Illustrative Embodiment 32: The system of illustrative embodiment 31, wherein the at least one processor is configured to execute the instructions to:
generate a first time for a first pulse of the plurality of pulses based on sampling a system time; and
generate a second time for a second pulse of the plurality of pulses based on the first time and the time offset.

Illustrative Embodiment 33: The system of any of illustrative embodiments 31-32, wherein the at least one processor is configured to execute the instructions to generate image measurement data based on the energy value for each of the plurality of pulses and the time offset value characterizing the time offset between the plurality of pulses.

Illustrative Embodiment 34: The system of illustrative embodiment 33, wherein the image measurement data is positron emission tomography (PET) measurement data.

Illustrative Embodiment 35: The system of any of illustrative embodiments 29-33, wherein the at least one processor is configured to execute the instructions to:
read model parameters from a memory device;
execute a machine learning model based on the model parameters; and
input the sampled signal data to the executed machine learning model.

Illustrative Embodiment 36: The system of illustrative embodiment 35, wherein the at least one processor is configured to execute the instructions to:
generate synthetic signals;
train the machine learning model based on the synthetic signals;
read the model parameters from the trained machine learning model; and
store the model parameters in the memory device.

Illustrative Embodiment 37: The system of illustrative embodiment 36, wherein the machine learning model is a Random Forrest model.

Illustrative Embodiment 38: The system of any of illustrative embodiments 29-37, wherein the plurality of pulses consists of two pulses.

Illustrative Embodiment 39: The system of any of illustrative embodiments 38, wherein the at least one processor is configured to execute the instructions to receive the at least one signal from a scanner of an image scanning system.

Illustrative Embodiment 40: The system of any of illustrative embodiments 29-39, wherein the at least one signal comprises a first signal that characterizes energy levels of the detection event.

Illustrative Embodiment 41: The system of illustrative embodiment 40, wherein the at least one signal comprises a second signal that characterizes a first dimension location of a crystal that detected the detection event.

Illustrative Embodiment 42: The system of illustrative embodiment 41, wherein the at least one signal comprises a third signal that characterizes a second dimension location of the crystal that detected the detection event.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein can be applied to other embodiments without the use of inventive faculty. The present disclosure is not intended to be limited to the embodiments shown herein, but is to be accorded the widest scope consistent with the principles and novel features disclosed herein.

What is claimed is:

1. A computer-implemented method comprising:
receiving at least one signal characterizing a detection event;
generating sampled signal data based on sampling the at least one signal;
applying a trained machine learning process to the sampled signal data and, based on the application of the trained machine learning process, generating pulse data characterizing a plurality of pulses; and
transmitting the pulse data characterizing the plurality of pulses.

2. The computer-implemented method of claim 1, wherein the pulse data comprises an energy value for each of the plurality of pulses.

3. The computer-implemented method of claim 2, wherein the pulse data comprises a time offset value characterizing a time offset between the plurality of pulses.

4. The computer-implemented method of claim 3, further comprising:
generating a first time for a first pulse of the plurality of pulses based on sampling a system time; and
generating a second time for a second pulse of the plurality of pulses based on the first time and the time offset.

5. The computer-implemented method of claim 3, further comprising generating image measurement data based on the energy value for each of the plurality of pulses and the time offset value characterizing the time offset between the plurality of pulses.

6. The computer-implemented method of claim 5, wherein the image measurement data is positron emission tomography (PET) measurement data.

7. The computer-implemented method of claim 1, wherein applying the trained machine learning process to the sampled signal data comprises:
reading model parameters from a memory device;
executing a machine learning model based on the model parameters; and
inputting the sampled signal data to the executed machine learning model.

8. The computer-implemented method of claim 7, further comprising:
generating synthetic signals;
training the machine learning model based on the synthetic signals;
reading the model parameters from the trained machine learning model; and
storing the model parameters in the memory device.

9. The computer-implemented method of claim 8, wherein the machine learning model is a Random Forrest model.

10. The computer-implemented method of claim 1, wherein the plurality of pulses consists of two pulses.

11. The computer-implemented method of claim 1, further comprising receiving the at least one signal from a scanner of an image scanning system.

12. The computer-implemented method of claim 1, wherein the at least one signal comprises a first signal that characterizes energy levels of the detection event.

13. The computer-implemented method of claim 12, wherein the at least one signal comprises a second signal that characterizes a first dimension location of a crystal that detected the detection event.

14. The computer-implemented method of claim 13, wherein the at least one signal comprises a third signal that characterizes a second dimension location of the crystal that detected the detection event.

15. A non-transitory computer readable medium storing instructions that, when executed by at least one processor, cause the at least one processor to perform operations comprising:
receiving at least one signal characterizing a detection event;
generating sampled signal data based on sampling the at least one signal;
applying a trained machine learning process to the sampled signal data and, based on the application of the trained machine learning process, generating pulse data characterizing a plurality of pulses; and
transmitting the pulse data characterizing the plurality of pulses.

16. The non-transitory computer readable medium of claim 15, wherein the pulse data comprises an energy value for each of the plurality of pulses and a time offset value characterizing a time offset between the plurality of pulses, and wherein the instructions, when executed by the at least one processor, further cause the at least one processor to perform operations comprising:
generating a first time for a first pulse of the plurality of pulses based on sampling a system time; and
generating a second time for a second pulse of the plurality of pulses based on the first time and the time offset.

17. The non-transitory computer readable medium of claim 16, wherein the instructions, when executed by the at least one processor, further cause the at least one processor to perform operations comprising generating image measurement data based on the energy value for each of the plurality of pulses and the time offset value characterizing the time offset between the plurality of pulses.

18. A system comprising:
a memory device storing instructions;
a transceiver; and
at least one processor communicatively coupled to the transceiver and to the memory device, the at least one processor configured to execute the instructions to:
receive, via the transceiver, at least one signal characterizing a detection event;
generate sampled signal data based on sampling the at least one signal;
apply a trained machine learning process to the at least one signal and, based on the application of the trained machine learning process, generate pulse data characterizing a plurality of pulses; and
transmit the pulse data characterizing the plurality of pulses.

19. The system of claim 18, wherein the pulse data comprises an energy value for each of the plurality of pulses and a time offset value characterizing a time offset between the plurality of pulses, and wherein the at least one processor is configured to execute the instructions to:
generate a first time for a first pulse of the plurality of pulses based on sampling a system time; and
generate a second time for a second pulse of the plurality of pulses based on the first time and the time offset.

20. The system of claim 19, wherein the at least one processor is configured to execute the instructions to generate image measurement data based on the energy value for each of the plurality of pulses and the time offset value characterizing the time offset between the plurality of pulses.
